(19) United States
(12) Patent Application Publication
(10) Pub. No.: US 2006/0193845 A1
(43) Pub. Date: Aug. 31, 2006

(54) Combination therapy for controlled carbohydrate digestion

(75) Inventors: Alan D. Watson, Lexington, MA (US); Laura Brass, Newton, MA (US); Bard J. Geesman, Cambridge, MA (US); Vaughn Kailian, Bodega, CA (US)

Correspondence Address: FISH & RICHARDSON PC, P.O. BOX 1022, MINNEAPOLIS, MN, 55440-1022, US

(73) Assignee: ELIXIR PHARMACEUTICALS, INC.

(21) Appl. No.: 11/227,696

(22) Filed: Sep. 14, 2005

Related U.S. Application Data
(60) Provisional application No. 60/610,126, filed on Sep. 14, 2004.

(51) Int. Cl. A61K 38/47; A61K 31/7034; A61K 31/695

(52) U.S. Cl. 424/94.61; 514/25; 514/63

(57) ABSTRACT

Carbohydrate usage in the gastro-intestinal tract of a subject can be modulated by administering, to a subject, a first agent that inhibits carbohydrate degradation in combination with a second agent that decreases formation or severity of intestinal gas.

### CROSS-REFERENCE TO RELATED APPLICATIONS

This application claims priority to U.S. Application Ser. No. 60/610,126, filed on Sep. 14, 2004.

### DESCRIPTION

Alpha glucosidase inhibitors are compounds that inhibit digestive enzymes such as amylase, sucrase, maltase, and α-dextrinase, to reduce the digestion of starch and sugars. These enzymes catalyze the decomposition of disaccharides in the intestine to monosaccharides. By slowing this process, alpha glucosidase inhibitors reduce acute post-prandial hyperglycemia. Examples of alpha glucosidase inhibitors include acarbose, miglitol, and voglibose (N-1(1,3-dihydroxy-2-propyl)valiolamine, and N-substituted derivatives thereof (see, e.g., U.S. Pat. No. 5,004,838), and N-substituted pseudo-amino sugars (see, e.g., U.S. Pat. No. 4,595,678)).

Acarbose is an inhibitor of the glucosidase class of enzymes in the small intestine as well as an inhibitor of pancreatic alpha amylase. Acarbose is 0-4,6-didesoxy-4-[(1S,4R,5S,6S)-4,5,6-trihydroxy-3-(hydroxymethyl)-2-cyclo hexen-1-yl amino]-α-D-glucopyranosyl-(1→4)-O-α-D-glucopyranosyl(1→4)-D-glucopyranose. The inhibitor can be obtained by fermentation of Actinoplanes species (see German Patent Specification 2,209,832, German Patent Specification 2,209,834, German Patent Specification 2,064,092) and can be isolated from the fermentation broth. Purification processes have been described for this purpose (see German Patent Specification 2,347,782 and German Patent Specification 2,719,912). U.S. Pat. No. 4,904,769 describes a method for preparing a highly purified preparation of acarbose. See also, e.g., U.S. Pat. No. 6,150,568.

By inhibiting glucosidases, acarbose delays digestion of complex carbohydrates and the subsequent absorption of glucose, resulting in a smaller rise in blood glucose concentration following meals. Acarbose can decrease post-prandial glucose (PPG) spikes. However, acarbose and other alpha glucosidase inhibitors can have side effects, including flatulence and abdominal pain.

Acarbose treatment has gastrointestinal (GI) side effects, including flatus, abdominal pain, and diarrhea. Such side effects are experienced by many patients, particularly at the initiation of therapy and at higher doses. The side effects are likely caused when excess undigested carbohydrates enter the lower part of the intestine, are not entirely broken down by the enzymes in the lower intestine, and so are metabolized by bacteria, producing carbon dioxide.

The side effects can be modulated to some extent by starting at a low dose and slowly titrating the dose upward, e.g., during the course of multiple months. Additionally, at least some side effects can subside over time because the patient's endogenous enzyme levels at lower parts of the intestine are upregulated, resulting in fewer carbohydrates being available to the gut flora. This reduction in the severity of the side effects does not affect the efficacy of acarbose because ingested carbohydrates are still delayed from entering the blood stream. Further reducing the side effects would alleviate social and physical discomfort, for example, during the interval in which the acarbose dose is being titrated, an interval which may span several weeks to months before a patient is at the desired dose. Reducing side effects could also improve the efficacy of acarbose on an “intention to treat” basis by increasing compliance.

Disclosed herein are a variety of methods for alleviating side effects of an alpha glucosidase inhibitor (e.g., acarbose or another agent that inhibits carbohydrate degradation, e.g., alpha-glucosidase activity). The methods generally include administering the alpha glucosidase inhibitor (e.g., acarbose or other alpha glucosidase inhibitor),e.g., an effective amount of the inhibitor, e.g., in combination with a second agent. For example, the second agent decreases formation or severity of intestinal gas.

Acarbose or other inhibitors of alpha-glucosidase activity can be used, e.g., in combination with the second agent, to treat or prevent a metabolic disorder, e.g., metabolic syndrome (e.g., Syndrome X), obesity, diabetes, etc. As used herein, a “metabolic disorder” refers to a disorder in which one skilled in the art would detect a physiological change in the subject that alters metabolism of at least one substance, e.g., carbohydrates or fats.

A metabolic syndrome (e.g., Syndrome X and syndrome-associated insulin resistance) is manifested in a patient who presents a group of metabolic risk factors. These factors include: central obesity (excessive fat tissue in and around the abdomen), atherogenic dyslipidemia (blood fat disorders—mainly high triglycerides and low HDL cholesterol—that foster plaque buildups in artery walls); insulin resistance or glucose intolerance (e.g., the body cannot properly use insulin or blood sugar); prothrombotic state (e.g., high fibrinogen or plasminogen activator inhibitor [−1] in the blood); raised blood pressure (130/85 mmHg or higher); and proinflammatory state (e.g., elevated high-sensitivity C-reactive protein in the blood). Overweight/obesity, physical inactivity and genetic factors can contribute to the syndrome. People with a metabolic syndrome are at increased risk of coronary heart disease, other diseases related to plaque buildups in artery walls (e.g., stroke and peripheral vascular disease) and type 2 diabetes. Metabolic syndrome can be closely associated insulin resistance.

In one embodiment, the metabolic disorder is diabetes, e.g., type 2 diabetes mellitus. For example, the patients can be normal (e.g., with respect to blood glucose levels), have impaired glucose tolerance (IGT), so-called pre-diabetic subjects, or diabetic subjects. The patients can have fasting hyperglycemia, e.g., patients that do not otherwise have diabetic characteristics and with fasting glucose levels between 100-125 mg/dL.

The invention provides methods of treating and preventing diabetes. Examples of diabetes include insulin dependent diabetes mellitus and non-insulin dependent diabetes. For example the method includes administering to a patient having diabetes or at risk of diabetes a compound described herein. In some instances, a patient can be identified as being at risk of developing diabetes by having impaired glucose tolerance (IGT), or fasting hyperglycemia.

For example, a compound described herein (e.g., an alpha glucosidase inhibitor) can be administered to a subject in a therapeutically effective amount to decrease gluconeogenesis, improve glycemic control (e.g., lower fasting blood glucose), or normalize insulin sensitivity. The compound can be administered to a subject suffering from diabetes or obesity.

Insulin dependent diabetes mellitus (Type 1 diabetes) is an autoimmune disease, where insulitis leads to the destruction of pancreatic J-cells. At the time of clinical onset of type 1 diabetes mellitus, significant number of insulin producing beta cells are destroyed and only 15% to 40% are still capable of insulin production (McCulloch et al. (1991) Diabetes 40:673-679). Beta cell failure results in a life long dependence on daily insulin injections and exposure to the acute and late complication of the disease.

Type 2 diabetes mellitus is a metabolic disease of impaired glucose homeostasis characterized by hyperglycemia, or high blood sugar, as a result of defective insulin action which manifests as insulin resistance, defective insulin secretion, or both. A patient with Type 2 diabetes mellitus has abnormal carbohydrate, lipid, and protein metabolism associated with insulin resistance and/or impaired insulin secretion. The disease leads to pancreatic beta cell destruction and eventually absolute insulin deficiency. Without insulin, high glucose levels remain in the blood. The long term effects of high blood glucose include blindness, renal failure, and poor blood circulation to these areas, which can lead to foot and ankle amputations. Early detection is critical in preventing patients from reaching this severity. The majority of patients with diabetes have the non-insulin dependent form of diabetes, currently referred to as Type 2 diabetes mellitus.

Acarbose or other inhibitors of alpha-glucosidase activity can be used, in combination with the second agent, to treat or prevent a large vessel disorder, e.g., atherosclerosis, stroke, peripheral vascular disease, myocardial infarction, and renal-vascular disease. For example, the method can be used in a variety of subjects, e.g., in normal subjects, in subjects with a genetic predisposition for the disorder, or in subjects who have a symptom or medical history indicative of the disorder, e.g., subjects who have had a heart attack or who have been diagnosed with the disorder.

In one aspect, the disclosure features a method that includes: administering, to a subject, a first agent that inhibits carbohydrate degradation (e.g., saccharidase activity) in combination with a second agent that decreases formation or severity of intestinal gas. The method can modulate carbohydrate usage in the gastro-intestinal tract of the subject. Typically the subject is a human subject. Typically, the second agent is administered in a manner such that the second agent acts preferentially in a specific part of the intestine, such as the ileum.

As used herein, “administered in combination” means that two or more agents are administered to a subject at the same time or within an interval, such that there is overlap of an effect of each agent on the patient. Preferably the administrations of the first and second agent are spaced sufficiently close together such that a combinatorial effect is achieved. The interval can be an interval of minutes, hours, days or weeks. Generally, the agents are concurrently bioavailable, e.g., detectable, in the subject. The first and second agents can be administered in either order. In a preferred embodiment at least one administration of one of the agents, e.g., the first agent, is made within minutes, one, two, three, or four hours, or even within one or two days of the other agent, e.g., the second agent. In some cases, combinations can achieve synergistic results, i.e., greater than additive results, e.g., at least 20, 50, 70, or 100% greater than additive.

For some embodiments, it is particularly advantageous to formulate the two agents together, e.g., in a single pill (e.g., tablet or gel). The use of a single pill that provides an adequate dose (e.g., for an adult or child) can increase compliance and ease administration.

In one embodiment, the first and second agents are administered at the same time. For example, the first and second agents are co-formulated. In another embodiment, the first and second agents are administered at different times. For example, the first agent can be administered prior to or during a meal, e.g., with the initial bite, and the second agent can be administered subsequent to a meal.

The first and second agents can be administered together in conjunction with each meal, e.g., prior to each meal, e.g., about two, three, or four times a day, or as required or at regular intervals.

In one embodiment, the first agent is an inhibitor of a glucosidase, e.g., alpha-glucosidase. In one embodiment, the first agent is acarbose or a related compound.

For example, the first agent includes:

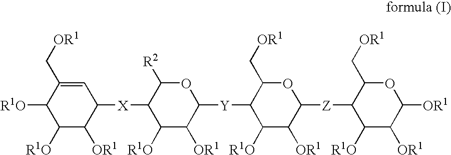

Each R¹ is independently H, C₁-C₆ alkyl, C(O)R³, or arylalkyl;

R² is C₁-C₆ alkyl;

each R³ is independently C₁-C₆ alkyl or aryl,

each X, Y, and Z is independently NR⁴ or O; and

each R⁴ is independently H, alkyl, or arylalkyl.

In some preferred embodiments, X is NR⁴, for example, NH. In some preferred embodiments Y and Z are O. In some preferred embodiments at least 3 R¹ moieties are H, for example, each R¹ is H. In some preferred embodiments, R² is methyl.

Examples of preferred alkyl moieties include methyl, ethyl, and propyl. Examples of preferred arylalkyl moieties include benzyl and phenylethyl. An example of a preferred C(O)R³ moiety includes acetyl.

The term “alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. The term “arylalkyl” refers to alkyl substituted with an aryl. The term “aryl” refers to a 6-carbon monocyclic, 10-carbon bicyclic, or 14-carbon tricyclic aromatic ring system wherein 0, 1, 2, 3, or 4 atoms of each ring may be substituted by a substituent. Examples of aryl groups include phenyl, naphthyl and the like.

In one embodiment, the first agent is voglibose. Voglibose, a disaccharide, is an intestinal alpha-glucosidase inhibitor.

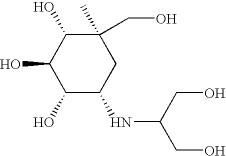

In one embodiment, the first agent is miglitol. Miglitol is a desoxynojirimycin derivative, and is chemically known as 3,4,5-piperidinetriol, 1-(2-hydroxyethyl)-2-(hydroxymethyl)-, [2R-(2α,3β,4α,5β)]. It can be prepared from a white to pale-yellow powder. It has a molecular weight of 207.2. Miglitol is soluble in water and has a pKa of 5.9. Its empirical formula is C₈H₁₇NO₅.

In another embodiment, the first agent is a compound extracted from a naturally occurring source, such as a Salacia plant (e.g., Salacia prinoides, Salacia reticulata, or Salacia oblonga). For example, the first agent is:

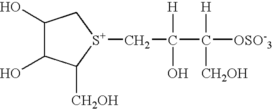

(salacinol), or:

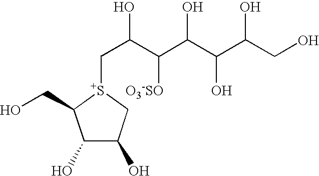

(kotalanol). These compounds are representative naturally occurring alpha glucosidase inhibitors. In other examples, the compound can be a compound extracted from Cinnamomum zeylanicum, Artocarpus heterophyllus, Tinospora cordifolia, or Pterocarpus marsupium.

In one embodiment, the second agent includes an enzyme that digests carbohydrate. A plurality of different second agents can be administered, e.g., a mixture of enzymes that digest carbohydrate. Exemplary enzymes include an alpha galactosidase, an alpha glucosidase, and a beta glucosidase. A representative example is BEANO®.

In another embodiment, the second agent includes or can be an anti-foaming agent, e.g., simethicone. Exemplary anti-foaming agents are ones that are not absorbed by the intestine.

The second agent can be formulated to preferentially deliver the second agent to the distal region of the colon. For example, the second agent can be formulated as a time delayed-release composition or a location-dependent release composition. Examples of location-dependent release compositions include pH sensitive formulations and enzyme triggered-formulations. In one implementation, the second agent is formulated by enteric encapsulation.

The subject can be a subject with normal or abnormal characteristics, e.g., with respect to a metabolic characteristic, e.g., normal or abnormal glucose tolerance. For example, the subject can have normal blood glucose response. In another example, the subject is glucose intolerant relative to the norm or has IGT. In another example, the subject has or is at risk for diabetes, e.g., type II diabetes mellitus. In still another example, the subject has or is at risk for a large vessel disorder or a metabolic disorder. The subject is generally a human, e.g., a human adult or child.

In another aspect, the disclosure features a method of administering acarbose to a subject. The method includes: administering, to the subject, acarbose in combination with an agent that decreases formation or severity of intestinal gas, the agent being administered in a manner such that the agent functions preferentially in intestine or colon.

Examples of an agent that decreases formation of intestinal gas include enzymes that digest carbohydrate. A plurality of different second agents can be administered, e.g., a mixture of enzymes that digest carbohydrate. Exemplary enzymes include an alpha galactosidase, alpha-glucosidase, and a beta-glucosidase. Examples of an agent that decreases severity of intestinal gas include anti-foaming agents, e.g., simethicone.

The agent can be formulated to preferentially deliver the agent to the distal region of the colon. For example, the agent is formulated as a time delayed-release composition or a location-dependent release composition. Examples of location-dependent release compositions include pH sensitive formulations and enzyme triggered-formulations. In one implementation, the second agent is formulated by enteric encapsulation.

In one embodiment, the agent that decreases formation of intestinal gas is administered subsequent to a meal, whereas acarbose is delivered prior to the meal.

In another aspect, the disclosure features a method of administering acarbose to a subject. The method includes: administering, to the subject, acarbose in combination with an anti-foaming agent. The subject can be administered acarbose and the anti-foaming agent prior to each major meal for an interval, e.g., of at least 10, 20, 30, or 50 days. In one embodiment, the dose of acarbose is increased in one or more increments during the interval, e.g., during the initial 30 days. In one embodiment, the dose of the anti-foaming agent is decreased in one or more decrements during the interval, e.g., during the initial 30 days. After the interval, e.g., the initial 30 days in which the acarbose and the anti-foaming agent are administered, acarbose is administered without the anti-foaming agent.

In another aspect, the disclosure features a method of modulating blood glucose levels. The method includes: administering, to a subject, acarbose in combination with an agent that decreases formation or severity of intestinal gas, the agent being administered in a manner such that the agent functions preferentially in intestine or colon.

In another aspect, the disclosure features a method of treating or preventing diabetes or a diabetes-related disorder. The method includes: administering, to a subject having diabetes, IGT, or fasting hyperglycemia, acarbose in combination with an agent that decreases formation or severity of intestinal gas, the agent being administered in a manner such that the agent functions preferentially in intestine or colon.

In another aspect, the disclosure features a method of treating or preventing a large vessel disorder, e.g., stroke, myocardial infarction, or peripheral vascular disease. The method includes: administering, to a subject, an alpha glucosidase inhibitor (e.g., acarbose) in combination with an agent that decreases formation or severity of intestinal gas, the agent being administered in a manner such that the agent functions preferentially in intestine or colon, whereby at least one symptom or predisposition of the large vessel disorder is ameliorated. Exemplary agents include a sugar cleaving enzyme (e.g., an alpha-galactosidase or beta-glucosidase) and an anti-foaming agent, e.g., simethicone.

In another aspect, the disclosure features a pharmaceutical preparation that includes: a first agent that inhibits alpha glucosidase activity; and a second agent that decreases formation or severity of intestinal gas. For example, the first agent is acarbose. Examples of the second agent includes a sugar cleaving enzyme (e.g., an alpha-galactosidase or beta-glucosidase), an agent aids expulsion of gas from the gastro-intestinal tract, or an anti-foaming agent. The preparation can be liquid, semi-solid, or solid. Examples include a tablet or gel.

The alpha-galactosidase enzyme can be from a non-human organism, e.g., a non-mammalian organism, e.g., from Aspergillus niger. Mammalian, e.g., human, enzymes can also be used. An exemplary composition is BEANO®, a mixture of about four enzymes. Exemplary components can include one or more of xylitol, invertase, disodium citrate, gelatin, and potassium sorbate.

The anti-foaming agent can be a silicone based antifoam. Exemplary anti-foaming agents include: ANTIFOAM FG-10™ made by Dow Corning, compositions containing a hydrocarbon-silicon copolymer, a hydrophobic filler, an organo-silicone surfactant, a hydrocarbon carrier oil, and, optionally, a silicone oil (see, e.g., U.S. Pat. No. 4,514,319), compositions comprising mineral oil-containing dispersed hydrophobic solid particles; hydrophobic silica in fluid hydrocarbon oil (see, e.g., U.S. Pat. No. 3,714,068); compositions comprising polyoxyethylene-polypropylene copolymers containing dispersed hydrophobic silica (see, e.g., U.S. Pat. No. 3,959,176); compositions containing a non-silicone water insoluble polyalkylene containing an alkoxysilicon chloride as the hydrophobic agent (see, e.g., G.B. Patent No. 1,166,877); compositions containing finely divided polyolefin polymers or polyesters dispersed in organic liquids (see, e.g., U.S. Pat. No. 3,705,859); compositions containing silicone oil-silica compounds and organo silicone compounds (see, e.g., U.S. Pat. No. 3,691,091); and compositions containing silicone-glycol copolymers in association with silicone oil and silica (see, e.g., U.S. Pat. No. 3,865,544). U.S. Pat. No. 5,458,886 describes some useful anti-foaming agents, including ones that contain titanium dioxide. The preparation can also include one or more of: calcium silicate and a water-soluble agglomerated maltodextrin. See, e.g., U.S. Pat. No. 5,073,384.

An anti-foaming agent can include, e.g., a mixture of from 92 to 98 percent by weight of one or more polydimethylsiloxanes and from 2 to 8 percent by weight of a high surface area silica (at least 50 m²/g).

An exemplary anti-foaming agent is simethicone. Simethicone is described in the NATIONAL FORMULARY, 14th Edition, American Pharmaceutical Association, Washington, D.C., 1975, at page 648, as a mixture of not less than 93% and not more than 99% of dimethylpolysiloxane and not less than 4% and not more than 4.5% of silicon dioxide. Other characteristics of simethicone are described in the aforementioned publication at the page indicated, and that description is incorporated herein by reference. Dimethylpolysiloxane is sometimes referred to as polysiloxane or organopolysiloxane. Related mixtures can also be used, e.g., another mixture of polydimethylsiloxane and a high surface area silica.

Simethicone can be present at various ratios with respect to the first agent (e.g., acarbose), e.g., on a weight-to-weight basis of about 10:1 to 2:1 or 1:1 or about 1:2 to 1:7.

In one embodiment, the preparation is formulated as a tablet, gel, or other fashion suitable for ingestion. For example, the first and second agent are partitioned from one another in the tablet. The second agent can be contained in an inner layer and the first agent can be contained in an outer layer. The preparation can contain between 10-20 mg (e.g., about 12.5 mg), 20-40 mg (e.g., about 25 mg), 40-65 mg (e.g., about 50 mg), or 80-120 mg (e.g., 100 mg) of the first agent. U.S. Pat. No. 5,456,920 describes an exemplary method for producing a tablet.

In another aspect, the disclosure features a pharmaceutical preparation that includes: a sugar cleaving enzyme in a controlled release formulation. For example, the enzyme has alpha galactosidase activity. The enzyme can be in crystalline form and/or may be crosslinked. In one embodiment, the enzyme is in an enteric coating, a pH sensitive formulation, or a time delayed release formulation. In one embodiment, the enzyme is a mutated enzyme with an altered pH sensitivity profile relative to wild-type. In one embodiment, the enzyme is formulated as a zymogen.

In another aspect, the disclosure features a kit that includes: a pharmaceutical composition including a first agent that inhibits alpha glucosidase activity, e.g., acarbose; and a pharmaceutical composition including a second agent that decreases formation or severity of intestinal gas (e.g., one or more carbohydrate digesting enzyme or simethicone). In another aspect, the disclosure features a kit that includes: a plurality of compartments, each including one or more units of a pharmaceutical composition. A first subset of the compartments of the plurality include units of the composition at a first dosage, and a second subset of the compartments of the plurality include units of the composition at a second dosage. The pharmaceutical composition includes a first agent that inhibits carbohydrate digestion and a second agent that decreases formation or severity of intestinal gas.

The dosage of active components in a pharmaceutical composition may be appropriately determined with reference to the dosages recommended for the respective active components and can be selected appropriately according to the recipient, the recipient's age and body weight, current clinical status, administration time, dosage form, method of administration, and combination of the active components, among other factors. The frequency of administration can be about one, two, three, or four times a day. The proportions of the active components in a pharmaceutical composition can be appropriately selected according to the recipient, the recipient's age and body weight, current clinical status, administration time, dosage form, method of administration, and combination of active components, among other factors. For example, voglibose can be used in a proportion of usually about 0.0001 to 0.2 weight parts, e.g., about 0.001 to 0.02 weight parts relative to 1 weight part of the compound or a salt thereof.

Acarbose is preferably administered such that it decreases carbohydrate degradation, e.g., glucosidase activity, in the proximal part of the colon. However, it is useful to retain the ability to digest carbohydrates (e.g., using glucosidase activity) in the distal part of the colon. Thus, carbohydrate would be less available to bacterial flora in the distal part of the colon.

A first agent (e.g., the agent that decreases carbohydrate degradation, e.g., acarbose) and/or the second agent can be formulated in a variety forms to control release of one or both of the agents, either separately or in combination. The second agent can be a compound (e.g., a commercially available compound) for decreasing intestinal gas. Particular examples include BEANO® and simethicone.

Any formulation can be adapted for formulating one or both of the first and second agents. For example, one of the formulations described in the following patent documents can be so adapted: U.S. Pat. No. 4,863,744 describes a delivery system for delivering an agent to a selected environment of use having a pH of greater than 3.5, e.g., a gastro-intestinal location after the stomach. US 2004-0062804 describes modified release dosage forms, including a slow release form for simethicone. US 2003-0108743 describes methods for intestinal release of an agent. U.S. Pat. No. 5,637,319 describes oral controlled-release preparations for drug delivery to various sites in the gastrointestinal tract, including the lower part of the intestine or colon.

In one embodiment, the formulation includes an enteric coating. U.S. Pat. No. 5,840,332 describes exemplary formulations for delivery of an agent to distal parts of the alimentary canal. The delivery system allows delivery to the duodenum, jejunum, ileum, ascending colon, transverse colon, and descending colon as the site for drug delivery. The low stomach pH and presence of gastric enzymes have led to the development of enteric coatings. Such coatings protect the gastric mucosa from drug irritation and protect drugs from inactivation by gastric enzymes and/or low pH. Coatings can be prepared using a selectively insoluble substance. Exemplary enteric coatings include methacrylic acid copolymers (EUDRAGITS™), cellulose acetate phthalate, cellulose acetate succinate, and styrol maleic acid co-polymers (Ritschel, W. A., Angewante Biopharmazie, Stuttgart (1973), pp. 396-402; Agyilirah, G. A., et al., “Polymers for Enteric Coating Applications” in Polymers for Controlled Drug Delivery, Tarcha, P. J. ed., CRC Press, (1991) Boca Raton, pp. 39-66).

In one embodiment, the second agent is a protein, e.g., a glucosidase or an galactosidase. The protein can be provided in a crystalline form, a cross-linked form, or combinations thereof. For example, U.S. Pat. No. 6,541,606 describes an exemplary stabilized protein crystals formulation. Another exemplary approach is crosslinked enzyme crystal technology. See, e.g., N. L. St. Clair et al., J. Am. Chem. Soc., 114, pp. 4314-16 (1992) and PCT/US91/05415. Crosslinked enzyme crystals can retain their activity in environments that are normally incompatible with enzyme function. Such environments include prolonged exposure to proteases, organic solvents, high temperature or extremes of pH. In such environments, crosslinked enzyme crystals remain insoluble, stable and active.

In general, crystals are produced by combining the protein to be crystallized with an appropriate aqueous solvent or aqueous solvent containing appropriate crystallization agents, such as salts or organic solvents. The solvent is combined with the protein and may be subjected to agitation at a temperature determined experimentally to be appropriate for the induction of crystallization and acceptable for the maintenance of protein activity and stability. The solvent can optionally include co-solutes, such as divalent cations, co-factors or chaotropes, as well as buffer species to control pH. The need for co-solutes and their concentrations are determined experimentally to facilitate crystallization.

Crosslinking may be carried out using reversible crosslinkers, in parallel or in sequence. The resulting crosslinked protein crystals are characterized by a reactive multi-functional linker, into which a trigger is incorporated as a separate group. The reactive functionality is involved in linking together reactive amino acid side chains in a protein and the trigger consists of a bond that can be broken by altering one or more conditions in the surrounding environment (e.g., pH, temperature, or thermodynamic water activity). The bond between the crosslinking agent and the protein may be a covalent or ionic bond, or a hydrogen bond. The change in surrounding environment results in breaking of the trigger bond and dissolution of the protein. Thus, when the crosslinks within protein crystals crosslinked with such reversible crosslinking agents break, dissolution of protein crystal begins and therefore the release of activity. Exemplary crosslinkers for crosslinking proteins in crystals are described in U.S. Pat. No. 6,541,606.

Protein crystals or formulations can themselves be encapsulated, e.g., in a polymeric coating to form a microsphere. The crystals are suspended in a polymeric carrier which is dissolved in an organic solvent. The polymer solution can be in an amount that provides a weight ratio of protein crystals to polymer between about 0.02 and about 20, preferably between about 0.1 and about 2. The protein crystals can be contacted with polymer in solution for a period of time between about 0.5 minutes and about 30 minutes, preferably between about 1 minutes and about 3 minutes.

Following that contact, the crystals become coated and are referred to as nascent microspheres. The nascent microspheres increase in size while coating occurs. In a preferred embodiment, the suspended coated crystals or nascent microspheres along with the polymeric carrier and organic solvent are transferred to a larger volume of an aqueous solution containing a surface active agent, known as an emulsifier. In the aqueous solution, the suspended nascent microspheres are immersed in the aqueous phase, where the organic solvent evaporates or diffuses away from the polymer. Eventually, a point is reached where the polymer is no longer soluble and forms a precipitated phase encapsulating the protein crystals or formulations to form a composition. The emulsifier can reduce the interfacial surface tension between the various phases of matter in the system during the hardening phase of the process. Alternatively, if the coating polymer has some inherent surface activity, there may be no need for addition of a separate surface active agent. Exemplary emulsifiers include poly(vinyl alcohol), surfactants and other surface active agents which can reduce the surface tension between the polymer coated protein crystals or polymer coated crystal formulations and the solution.

In one embodiment, the crystal is a crystal that includes at least one or more enzymes present in BEANO®.

Example: Administration of an Alpha-Glucosidase Inhibitors (Such as Acarbose) and a Carbohydrate-Cleaving Enzyme (CCE)

An alpha-glucosidase inhibitors (such as acarbose) can be administered in combination with an CCE, for example, at the beginning of a meal. The two agents can be administered to minimize the possibility of a cancellation effect, e.g., to prevent the inhibitor from inactivating the enzyme. Either or both agents can be formulated using a time delayed release formulation. For example, the agents can be formulated to have different release profiles, e.g., such that the profiles favor the inhibitor being released in the upper part of the intestine or the proximal colon and the CCE being released further down the GI tract, e.g., in the distal colon. This design would facilitate acarbose action in the upper part of the intestine to delay glucose absorption, and CCE action in the lower part of the intestine to prevent the excess carbohydrates from being digested by enteric bacteria. In one embodiment, the inhibitor is formulated for typical release whereas the CCE is formulated for time delayed release.

In another embodiment, the CCE is formulated for pH sensitive release, e.g., such that the CCE would not be released or activated until it hit a certain pH level characteristic of the lower part of the intestine. The release could be triggered by time, pH, other enzyme activation (i.e., the CCE-type agent would be bound up until enzymes or conditions in the lower intestine released or activated it), the dissolution of certain coatings on the molecule or pill, etc. It is also possible to mutate the CCE so that enzymatic activity is more pH sensitive, e.g., to decrease activity at acidic pH, but have greater relative activity at the less acidic pH of the distal colon. In one embodiment, the CCE is an enzyme available from BEANO®.

Example: Administration of an Alpha-Glucosidase Inhibitor (Such as Acarbose)+Simethicone

Simethicone or another anti-foaming agent can be used to ameliorate effects of excess gas in the intestinal tract. Anti-foaming agents can alleviate froth in the stomach or lower bowel, e.g., by facilitating expulsion of the gas by belching or passing flatus. Simethicone and other anti-foaming agents reduce surface tension and thereby disrupt or break bubbles. Simethicone, for example, is not absorbed from the intestine, nor is it known to have adverse side effects, e.g., with a condition or medication. Simethicone could be in a regular or controlled release formulation, e.g., a time delayed release or pH dependent release formulation.

Example

One implementation a features a package (e.g., a blister pack) that includes a plurality of compartments. Each compartment can include at least one unit dosage of a glucosidase inhibitor (e.g., acarbose). The compartments can be ordered, e.g., to have low dosages in one area of the package, medium doses in another area, and high doses in a third area. For example, the compartment can be presented sequentially, e.g., going left to right and then down, or going in a circle, e.g., clockwise. The compartments can be organized such that compartments early in the sequence have a low dose (e.g., 25 mg), compartments midway through the sequence have a second dose (e.g., 50 mg) and compartments later in the sequence have a third dose (e.g., 100 mg). The package can be used to provide a gradually increasing dosage of the glucosidase inhibitor.

Unit doses can be prepared for each of two or three meals anticipated during the diurnal cycle.

Example

A tablet is produced containing 25 mg acarbose and 300 GaIU BEANO®. It can be administered three times a day with meals. For example, it can be taken prior to meals.

Example

A tablet is produced containing 0.2 mg voglibose and 300 GaIU BEANO®. It can be administered three times a day with meals. For example, it can be taken prior to meals.

Other embodiments are within the following claims. All patents, applications, and references are hereby incorporated by reference in their entireties.

What is claimed is:

1. A method of modulating carbohydrate usage in the gastro-intestinal tract of a subject, the method comprising:
administering, to the subject, acarbose in combination with an agent that decreases formation or severity of intestinal gas.

2. The method of claim 1 in which acarbose and the agent are co-formulated.

3. The method of claim 1 in which the agent comprises an enzyme that digests carbohydrate or a mixture of enzymes that digest carbohydrate.

4. The method of claim 1 in which the agent is formulated as a delayed-release composition or a location-dependent release composition.

5. The method of claim 1 in the agent being administered in a manner such that the agent functions preferentially in intestine or colon.

6. The method of claim 1 in which the agent is formulated by enteric capsulation.

7. The method of claim 1 in which the agent is formulated as an enzyme triggered-release composition.

8. The method of claim 1 in which the agent comprises an anti-foaming agent.

9. The method of claim 1 in which the subject is has normal blood glucose response.

10. The method of claim 1 in which the subject is glucose intolerant relative to the norm or has impaired glucose tolerance.

11. The method of claim 1 in which the subject has or is at risk for diabetes, a large vessel disorder, or a metabolic syndrome.

12. A method of administering acarbose to a subject, the method comprising:
administering, to the subject, acarbose in combination with an anti-foaming agent.

13. The method of claim 12 in which the anti-foaming agent is simethicone.

14. The method of claim 12 in which the subject is administered acarbose and the anti-foaming agent prior to each major meal for at least 30 days.

15. The method of claim 14 in which the dose of acarbose is increased in one or more increments during the initial 30 days.

16. The method of claim 14 in which the dose of the anti-foaming agent is decreased in one or more decrements during the initial 30 days.

17. The method of claim 14 after an initial period in which the acarbose and the anti-foaming agent are administered, acarbose is administered without the anti-foaming agent.

18. A pharmaceutical preparation comprising:
acarbose; and
a second agent that decreases formation or severity of intestinal gas.

19. The pharmaceutical preparation of claim 18 wherein the second agent comprises a sugar cleaving enzyme.

20. The pharmaceutical preparation of claim 19 in which the sugar cleaving enzyme is alpha-galactosidase.

21. A pharmaceutical preparation comprising: acarbose and simethicone.
